(19) United States
(12) Patent Application Publication
(10) Pub. No.: US 2009/0318316 A1
(43) Pub. Date: Dec. 24, 2009

(54) LUBRICATING OIL COMPOSITION AND LUBRICATING OIL FOR FLUID DYNAMIC BEARING AS WELL AS FLUID DYNAMIC BEARING AND METHOD FOR LUBRICATING FLUID DYNAMIC BEARING USING THE SAME

(75) Inventor: Yoshiyuki Morishima, Toda-shi (JP)

Correspondence Address: OLIFF & BERRIDGE, PLC, P.O. BOX 320850, ALEXANDRIA, VA, 22320-4850, US

(73) Assignee: JAPAN ENERGY CORPORATION, TOKYO (JP)

(21) Appl. No.: 12/310,368

(22) PCT Filed: Aug. 23, 2007
(86) PCT No.: PCT/JP2007/066390

§ 371 Date: Feb. 23, 2009

(30) Foreign Application Priority Data
Sep. 13, 2006 (JP) 2006-248211

(51) Int. Cl. F16C 33/00; C07C 59/245; C10M 105/32; C10M 129/18; C10M 133/44

(52) U.S. Cl. 508/100; 554/213; 508/497; 508/304; 508/279

(57) ABSTRACT

This invention relates to a lubricating oil composition being excellent in oxidation stability and low-temperature flowability, and more particularly to a lubricating oil composition characterized by using as a base oil a diester obtained from a straight-chain dihydric alcohol having a carbon number of 6 to 12 and having a hydroxyl group at both terminal carbon atoms respectively and a branched-chain saturated monovalent fatty acid having a carbon number of 6 to 12.

### TECHNICAL FIELD

This invention relates to a lubricating oil composition and a lubricating oil for a fluid dynamic bearing composed of the lubricating oil composition, as well as a fluid dynamic bearing and a method for lubricating a fluid dynamic bearing using the lubricating oil composition, and more particularly to a lubricating oil composition having a high oxidation stability and an excellent low-temperature flowability and being suitable for industrial lubricating oils such as a bearing oil, a gear oil, a refrigerating machine oil, a turbine oil, a hydraulic oil, a compressor oil, a machine tool oil, a metal working oil and so on, as well as an automotive lubricating oil, a marine lubricating oil and the like.

### BACKGROUND ART

At present, a lubricating oil is widely used in various industries, and its action is to primarily decrease friction and wear on a contact face while metals are slid in contact with each other. A property required for the lubricating oil depends on an application, but generally includes lubricity, oxidation stability, thermal stability, low-temperature flowability, viscosity characteristics and so on. In order to satisfy these properties, heretofore, various natural products and synthetic products are used as a base oil and an additive of the lubricating oil composition. For example, as the natural product are used a mineral oil, animal and vegetable fats and oils, and a fatty acid derived from animal and vegetable oils, and as the synthetic product are used α-olefin oligomer, polyalkylene glycol, fatty acid monoester and diester, polyol (hindered) ester, phosphate ester, silicate ester, silane, silicone, polyphenylene ether, fluorocarbon and so on. By using them alone or in a combination, a lubricating oil having a target property is accomplished.

On the other hand, a use condition of the lubricating oil becomes severer with recent diversity and upgrading of industries, and thereby it is difficult to sufficiently satisfy a required property with a conventional synthetic lubricating oil. Concretely, the use condition becomes severer such that a working temperature is within an ultralow temperature range of not higher than −30° C. or a high temperature range of higher than 150° C. due to heating, sliding in contact with a metal or the like. In this context, there is recently required a lubricating oil which can be used under such a condition for a long time, and it is said that good lubricity, heat resistance, oxidation stability and low-temperature flowability as well as a high viscosity index is very important as a property of such a lubricating oil. As such a lubricating oil is proposed a lubricating oil using as a base oil dicarboxylic acid ester, an ether composed of a polyoxyalkylene glycol and an alcohol, or an ester composed of a polyoxyalkylene glycol and an aliphatic monovalent carboxylic acid (see JP-A-S62-263288).

Moreover, a fluid dynamic bearing comprising a sleeve and a rotating shaft which oppose to each other via a lubricating oil recently comes into use for a rotation device for driving a magnetic disc and an optical disc such as a FD, a MO, a zip, a mini-disc, a compact disc (CD), a DVD, a hard disc and so on in an electronic device such as image and acoustic devices, a personal computer and the like. In this regard, lubricity, degradation stability (lifetime), sludge-formation preventability, wear preventability, corrosion preventability and the like are commonly required for the lubricating oil used in the fluid dynamic bearing, and there are hitherto proposed one comprising at least one of an olefin-based, diester-based or neopentyl polyol ester-based synthetic oil, squalane and a naphthene-based mineral oil, as well as a grease of an urea compound-based thickener (see JP-A-H01-279117), one using a fatty acid triester of trimethylolpropane as a base oil and comprising a hindered phenol-based antioxidant and a benzotriazole derivative (see JP-A-H01-188592), one comprising a specific macromolecular hindered phenol-based antioxidant and an aromatic amine-based antioxidant at a specified ratio (see JP-A-H01-225697), one using a specified monocarboxylic acid ester and/or a specified dicarboxylic acid diester having a phenyl group as a base oil (see JP-A-H04-357318), one using a single composition as a base oil (see JP-B-2621329), one using a carbonic ester as a base oil and comprising a sulfur-containing phenol-based antioxidant and a zinc-based extreme pressure agent (see JP-A-H08-34987), one comprising a magnetic fluid (see JP-A-H08-259977, JP-A-H08-259982 and JP-A-H08-259985), one using a specified carbonic ester as a base oil and comprising a phenol-based antioxidant (see JP-A-H10-183159), one using as a base oil an ester of trimethylolpropane with a monovalent fatty acid having a carbon number of 4 to 8 (see JP-A-2004-091524), one using as a base oil a diester of pimeric acid and/or suberic acid with a branched-chain monohydric alcohol having a carbon number of 6 to 10 (see JP-A-2004-250625), one using as a base oil a diester of a dicarboxylic acid with an oxyalkylene alcohol (see JP-A-2006-096849) and so on.

### DISCLOSURE OF THE INVENTION

As mentioned above, it is required that the lubricating oil can be used under a severe condition for a long time, and in particular, the lubricating oil is required to be excellent in the lubricity, heat resistance, oxidation stability and low-temperature flowability and to have a high viscosity index. However, even the lubricating oil described in JP-A-S62-263288 cannot sufficiently satisfy these properties, and in particular, there is room for improvement in the oxidation stability and the low-temperature flowability.

Further, since the electronic device is recently popularized and tends to be used under a severe environment, and in particular, a device mounted and used in a vehicle such as a car navigation system and the like must wear in a cold region and under the blazing sun in view of a use environment of an automobile. Therefore, a lubricating oil for a fluid dynamic bearing is also required to be excellent in the oxidation stability and the low-temperature flowability in addition to the above-described various properties. However, the lubricating oils for the fluid dynamic bearing disclosed in JP-A-H01-279117, JP-A-H01-188592, JP-A-H01-225697, JP-A-H04-357318, JP-B-2621329, JP-A-H08-34987, JP-A-H08-259977, JP-A-H08-259982, JP-A-H08-259985, JP-A-H10-183159, JP-A-2004-091524, JP-A-2004-250625 and JP-A-2006-096849 do not have the sufficient oxidation stability and low-temperature flowability, and a further improvement is required.

It is, therefore, an object of the invention to solve the above-mentioned problems of the conventional techniques and to provide a lubricating oil composition being excellent in the oxidation stability and the low-temperature flowability. Also, it is another object of the invention to provide a lubricating oil for a fluid dynamic bearing composed of such a lubricating oil composition as well as a fluid dynamic bearing and a method for lubricating a fluid dynamic bearing using such a lubricating oil composition.

The inventor has made various studies in order to achieve the above objects and discovered that the oxidation stability, hydrolytic stability and the low-temperature flowability of the lubricating oil composition are improved by using as a base oil a diester of a straight-chain dihydric alcohol having a hydroxyl group at both terminals and a branched-chain saturated monovalent fatty acid, and as a result the invention has been accomplished.

That is, the lubricating oil composition according to the invention is characterized by using as a base oil a diester obtained from a straight-chain dihydric alcohol having a carbon number of 6 to 12 and having a hydroxyl group at both terminal carbon atoms respectively and a branched-chain saturated monovalent fatty acid having a carbon number of 6 to 12.

In the lubricating oil composition according to the invention, the dihydric alcohol is preferable to be 1,8-octanediol, 1,9-nonanediol or 1,10-decanediol, while the monovalent fatty acid is preferable to be 2-ethyl hexanoic acid or 3,5,5-trimethyl hexanoic acid.

The lubricating oil composition according to the invention preferably has a viscosity index of not lower than 100 and a pour point of not higher than −50° C.

The lubricating oil composition according to the invention preferably comprises 0.01 to 5% by mass of an amine-based antioxidant. In this case, the lubricating oil composition is more preferable to have a phenol-based antioxidant content of not more than 0.1% by mass. Moreover, the lubricating oil composition is more preferable to comprise at least one selected from the group consisting of an epoxy compound, a carbodiimide compound and a triazole compound in an amount of 0.01 to 2% by mass.

Further, the lubricating oil for the fluid dynamic bearing according to the invention is characterized by being composed of the above-described lubricating oil composition, the fluid dynamic bearing according to the invention comprises a shaft and a sleeve and is characterized in that the above-described lubricating oil composition is held in a gap between the shaft and the sleeve, and the method for lubricating the fluid dynamic bearing according to the invention is characterized by lubricating a gap between a shaft and a sleeve of the fluid dynamic bearing comprising the shaft and the sleeve by using the above-described lubricating oil composition.

According to the invention, there can be provided a lubricating oil composition using as a base oil a diester synthesized from a straight-chain dihydric alcohol having a hydroxyl group at both terminals and a branched-chain saturated monovalent fatty acid and having the excellent oxidation stability, hydrolytic stability and low-temperature flowability (particularly low-temperature flowability after a long-term storage). Further, there can be provided a lubricating oil for a fluid dynamic bearing composed of such a lubricating oil composition, as well as a fluid dynamic bearing and a method for lubricating a fluid dynamic bearing using such a lubricating oil composition.

### BRIEF DESCRIPTION OF THE DRAWINGS

FIG. 1 is a sectional view schematically showing an outline structure of a motor equipped with a fluid dynamic bearing.

### BEST MODE FOR CARRYING OUT THE INVENTION

<Lubricating Oil Composition>

The lubricating oil composition according to the invention will be described in detail below. The lubricating oil composition according to the invention is characterized by using as a base oil a diester obtained from a straight-chain dihydric alcohol having a carbon number of 6 to 12 and having a hydroxyl group at both terminal carbon atoms respectively and a branched-chain saturated monovalent fatty acid having a carbon number of 6 to 12. In the diester used as a base oil of the lubricating oil composition according to the invention, a portion derived from the fatty acid is branched and thereby the diester has a low pour point and dose not be solidified even if it is stored at low temperature for a long time. In this context, in the diester obtained from a branched-chain dihydric alcohol having a carbon number of 6 to 12 and a straight-chain saturated monovalent fatty acid having a carbon number of 6 to 12, a portion derived from the alcohol is branched, and thereby the diester has a low pour point but may be solidified if it is stored at low temperature for a long time. Further, although the reason is not necessarily clear, the lubricating oil composition according to the invention has a higher oxidation stability as compared with a lubricating oil composition using as a base oil a diester obtained from a straight-chain divalent carboxylic acid having a carbon number of 6 to 12 and having a carboxyl group at both terminals respectively and a branched-chain saturated monohydric alcohol having a carbon number of 6 to 12, particularly has a notably high improvement in the oxidation stability with an addition of an amine-based antioxidant.

The diester used for the lubricating oil composition according to the invention is synthesized by an esterification reaction of the straight-chain dihydric alcohol having a carbon number of 6 to 12 and having a hydroxyl group at both terminal carbon atoms respectively with the branched-chain saturated monovalent fatty acid having a carbon number of 6 to 12.

As the dihydric alcohol are mentioned 1,6-hexanediol, 1,7-heptanediol, 1,8-octanediol, 1,9-nonanediol, 1,10-decanediol, 1,11-undecanediol, 1,12-dodecanediol and the like. Among them, 1,8-octanediol, 1,9-nonanediol and 1,10-decanediol are preferable from a viewpoint of balancing low evaporativity and energy saving. These dihydric alcohols may be used alone or in a combination or two or more.

On the other hand, as the monovalent fatty acid are mentioned 2-methyl pentanoic acid, 2-ethyl pentanoic acid, 2-methyl hexanoic acid, 2-ethyl hexanoic acid, 3-ethyl hexanoic acid, 2-methyl heptanoic acid, 3,5,5-trimethyl hexanoic acid, isodecanoic acid and the like. Among them, 2-ethyl hexanoic acid and 3,5,5-trimethyl hexanoic acid are preferable from a viewpoint of balancing low evaporativity and low-temperature flowability, and 2-ethyl hexanoic acid is particularly preferable. These monovalent fatty acids may be used alone or in a combination or two or more.

Further, as the diester synthesized from the dihydric alcohol and the monovalent fatty acid are mentioned 1,8-bis(2-ethyl hexanoyloxy)octane, 1,9-bis(2-ethyl hexanoyloxy)nonane, 1,10-bis(2-ethyl hexanoyloxy)decane, 1,8-bis(3,5,5-trimethyl hexanoyloxy)octane, 1,9-bis(3,5,5-trimethyl hexanoyloxy)nonane, 1,10-bis(3,5,5-trimethyl hexanoyloxy)decane and the like. These diesters may be used alone or in a combination or two or more.

In the lubricating oil composition according to the invention, a content of the diester is preferably not less than 90% by mass, more preferably not less than 95% by mass. When the content of the diester is not less than 90% by mass, the oxidation stability and the low-temperature flowability of the lubricating oil composition can be sufficiently improved.

The lubricating oil composition according to the invention preferably comprises an amine-based antioxidant in an amount of 0.01 to 5% by mass as an additive, more preferably in an amount of 0.02 to 3% by mass, even more preferably in an amount of 0.05 to 2% by mass. When the content of the amine-based antioxidant is not less than 0.01% by mass, sufficient oxidation stability can be given to the lubricating oil composition, while when it is not more than 5% by mass, formation of sludge can be sufficiently suppressed.

As the amine-based antioxidant are mentioned (1) monoalkyl diphenylamines such as monooctyl diphenylamine, monononyl diphenylamine and so on, (2) dialkyl diphenylamines such as 4,4′-dibutyl diphenylamine, 4,4′-dipentyl diphenylamine, 4,4′-dihexyl diphenylamine, 4,4′-diheptyl diphenylamine, 4,4′-dioctyl diphenylamine, 4,4′-dinonyl diphenylamine and so on, (3) polyalkyl diphenylamines such as tetrabutyl diphenylamine, tetrahexyl diphenylamine, tetraoctyl diphenylamine, tetranonyl diphenylamine and so on, (4) naphthylamines and derivatives thereof such as 6 -naphthylamine, phenyl-α-naphthylamine, butylphenyl-α-naphthylamine, pentylphenyl-α-naphthylamine, hexylphenyl-α-naphthylamine, heptylphenyl-α-naphthylamine, octylphenyl-α-naphthylamine, nonylphenyl-α-naphthylamine and so on. Among them, the dialkyl diphenylamines and the alkylphenyl naphthylamines are preferable, the dialkyl diphenylamines and the alkylphenyl naphthylamines with an alkyl group having a carbon number of 4 to 24 are more preferable, and the dialkyl diphenylamines and the alkylphenyl naphthylamines with an alkyl group having a carbon number of 6 to 18 are even more preferable. These amine-based antioxidants may be used alone or in a combination or two or more.

The lubricating oil composition according to the invention may further comprise a phenol-based antioxidant in addition to the amine-based antioxidant. However, a content of the phenol-based antioxidant in the lubricating oil composition is preferably not more than 0.1% by mass, more preferably not more than 0.03% by mass, even more preferably not more than 0.01% by mass. Most preferably, the lubricating oil composition does not include the phenol-based antioxidant. When the content of the phenol-based antioxidant is not more than 0.1% by mass, more excellent oxidation stability can be given to the lubricating oil composition.

As the phenol-based antioxidant are mentioned 2,6-di-t-butylphenol, 2,6-di-t-butyl-4-methylphenol, 4,4′-methylene-bis(2,6-di-t-butylphenol), 4,4′-butylidene-bis(3-methyl-6-t-butylphenol), 2,2′-methylene-bis(4-ethyl-6-t-butylphenol), 2,2′-methylene-bis(4-methyl-6-t-butylphenol), 4,4′-isopropylidene-bisphenol, 2,4-dimethyl-6-t-butylphenol, tetrakis[methylene-3-(3,5-di-t-butyl-4-hydroxyphenyl)propionate]methane, 1,1,3-tris(2-methyl-4-hydroxy-5-t-butylphenyl)butane, 1,3,5-trimethyl-2,4,6-tris(3,5-di-t-butyl-4-hydroxybenzyl)benzene, 2,6-di-t-butyl-4-ethylphenol, 2,6-bis(2′-hydroxy-3′-t-butyl-5′-methylbenzyl)-4-methylphenol, bis[2-(2-hydroxy-5-methyl-3-t-butylbenzyl)-4-methyl-6-t-butylphenyl terephthalate, triethyleneglycol-bis[3-(3-t-butyl-5-methyl-4-hydroxyphenyl)propionate], and 1,6-hexanediol-bis[3-(3,5-di-t-butyl-4-hydroxyphenyl)propionate].

The lubricating oil composition according to the invention preferably further comprises at least one selected from the group consisting of an epoxy compound, a carbodiimide compound and a triazole compound in an amount of 0.01 to 2% by mass, more preferably in an amount of 0.02 to 1% by mass, in addition to the amine-based antioxidant. When the content of these compounds is not less than 0.01% by mass, the oxidation stability of the lubricating oil composition is further improved and also hydrolytic stability of the diester used as a base oil can be improved, while when it is not more than 2% by mass, formation of sludge can be sufficiently suppressed.

The epoxy compound preferably has a carbon number of 4 to 60, more preferably 5 to 25. In this regard, as the epoxy compound are concretely mentioned glycidyl ethers such as butyl glycidyl ether, 2-ethylhexyl glycidyl ether, trimethylolpropane polyglycidyl ether, neopentyl glycol diglycidyl ether, t-butylphenyl glycidyl ether and so on, glycidyl esters such as glycidyl adipate, glycidyl 2-ethylhexanoate, glycidyl isononanoate, glycidyl neodecanoate and so on, epoxidized fatty acid monoesters such as epoxidized methyl stearate and so on, and epoxidized vegetable oils such as epoxidized soybean oil and so on. Further, as the epoxy compound are preferable glycidyl ethers represented by the following general formula (I):

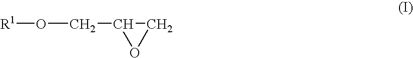

[wherein R¹ is a hydrogen atom, a straight-chain or branched-chain alkyl group having a carbon number of 1 to 24, or an alkylphenyl group having a carbon number of 7 to 24], glycidyl ethers represented by the following general formula (II):

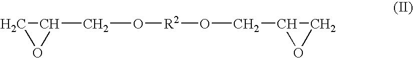

[wherein R² is a straight-chain or branched-chain alkylene group having a carbon number of 1 to 18], and glycidyl esters represented by the following general formula (III):

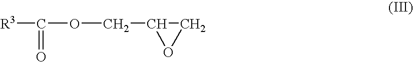

[wherein R³ is a straight-chain or branched-chain alkyl group having a carbon number of 1 to 24, or an alkylphenyl group having a carbon number of 7 to 24], and the glycidyl esters of the formula (1) are particularly preferable. These epoxy compounds may be used alone or in a combination or two or more.

The carbodiimide compound are preferably represented by the following general formula (IV):

R⁴—N═C═N—R⁵ (IV)

[wherein R⁴ and R⁵ are independently a hydrocarbon group having a carbon number of 1 to 24, preferably an alkylphenyl group having a carbon number of 7 to 24, more preferably an alkylphenyl group having a carbon number of 7 to 18]. As the carbodiimide compound are concretely mentioned 1,3-diisopropylcarbodiimide, 1,3-di-t-butylcarbodiimide, 1,3-dicyclohexylcarbodiimide, 1,3-di-p-tolylcarbodiimide, 1,3-bis(2,6-diisopropylphenyl)carbodiimide and so on. Among them, 1,3-diisopropylcarbodiimide, 1,3-di-p-tolylcarbodiimide and 1,3-bis(2,6-diisopropylphenyl)carbodiimide are preferable. These carbodiimide compounds may be used alone or in a combination or two or more.

As the triazole compound are mentioned benzotriazole and benzotriazole derivatives, and compounds represented by the following general formula (V):

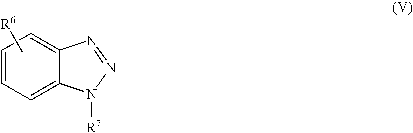

[wherein R⁶ is a hydrogen atom or methyl group, and R⁷ is a hydrogen atom or a nitrogen atom and/or oxygen atom-containing monovalent group having a carbon number of 0 to 20] are preferable. The benzotriazole derivatives are more preferable as the triazole compound, and compounds represented by the formula (V) wherein R⁷ is a nitrogen atom-containing monovalent group having a carbon number of 5 to 20 are even more preferable. As the triazole compound are concretely mentioned 2-(2′-hydroxy-5′-methylphenyl)benzotriazole, 2-[2′-hydroxy-3′,5′-bis(α,α′-dimethylbenzyl)phenyl]benzotriazole, 2-(2′-hydroxy-3′,5′-di-t-butylphenyl)benzotriazole, 1-[N,N-bis(2-ethylhexyl)aminomethyl]benzotriazole and so on. These triazole compounds may be used alone or in a combination or two or more.

The lubricating oil composition according to the invention may further comprise a detergent dispersant, an antiwear agent, a viscosity index improver, a pour point depressant, an ashless dispersant, a metal deactivator, a metal-based detergent, an oiliness agent, a surfactant, an antifoaming agent, a friction modifier, an antirust agent, a corrosion inhibitor and the like, if necessary.

The lubricating oil composition according to the invention preferably has a viscosity index of not lower than 100, more preferably not lower than 105, even more preferably not lower than 110. When the viscosity index of the lubricating oil composition is not lower than 100, a viscosity at ultralow temperature does not become abnormally high and thereby an increase in electrical power consumption can be suppressed. Moreover, the lubricating oil composition according to the invention preferably has a pour point of not higher than −50° C. from a viewpoint of the low-temperature flowability.

Preferably, the lubricating oil composition according to the invention does not crystallize even if it is stored at −40° C. for 30 days from a viewpoint of the low-temperature flowability after long-term storage. Also, the lubricating oil composition according to the invention preferably has a total acid number of not higher than 1 mgKOH/g, more preferably not higher than 0.3 mgKOH/g from a viewpoint of corrosion preventability, wear resistance and safety. Further, the lubricating oil composition according to the invention preferably has a hydroxyl value of not higher than 20 mgKOH/g, more preferably not higher than 5 mgKOH/g from a viewpoint of moisture-absorption resistance and safety. Furthermore, the lubricating oil composition according to the invention preferably has a specific inductive capacity at 25° C. of not lower than 2.5, more preferably 2.7 to 10, even more preferably 2.9 to 8.0.

<Lubricating Oil for the Fluid Dynamic Bearing, Fluid Dynamic Bearing and Method for Lubricating the Fluid Dynamic Bearing>

Then, the lubricating oil for the fluid dynamic bearing, the fluid dynamic bearing and the method for lubricating the fluid dynamic bearing according to the invention will be described in detail. The lubricating oil for the fluid dynamic bearing according to the invention is characterized by being composed of the above-mentioned lubricating oil composition. Also, the fluid dynamic bearing according to the invention comprises a shaft and a sleeve and is characterized in that the above-mentioned lubricating oil composition is held in a gap between the shaft and the sleeve. Further, the method for lubricating the fluid dynamic bearing according to the invention is characterized by lubricating a gap between a shaft and a sleeve of the fluid dynamic bearing comprising the shaft and the sleeve by using the above-mentioned lubricating oil composition. The fluid dynamic bearing according to the invention is not particularly limited mechanically, as far as it does not have a mechanism such as a ball bearing or the like but comprises the shaft and the sleeve, wherein the gap between the shaft and the sleeve is kept by the lubricating oil contained therebetween so as not to directly contact with each other. Further, the fluid dynamic bearing according to the invention includes a fluid dynamic bearing wherein a rotating shaft and/or a sleeve is provided with a dynamic pressure generating groove and the rotating shaft is supported by dynamic pressure, a fluid dynamic bearing provided with a thrust plate so as to generate dynamic pressure in a direction perpendicular to a rotating shaft, and the like.

In the fluid dynamic bearing, the sleeve is partially or wholly contacted with the rotating shaft or the thrust plate because the dynamic pressure is not generated during non-rotation, and the dynamic pressure generated by rotation makes the fluid dynamic bearing into non-contact state. Thus, the contact and the non-contact are repeated to probably cause metal wear between the sleeve and the rotating shaft or between the sleeve and the thrust plate, or temporary contact during rotation may cause seizure. However, high-speed rotation stability and durability are maintained for a long time by using the lubricating oil composition according to the invention being excellent in the oxidation stability and the low-temperature flowability, and the fluid dynamic bearing stably acts even at low temperature.

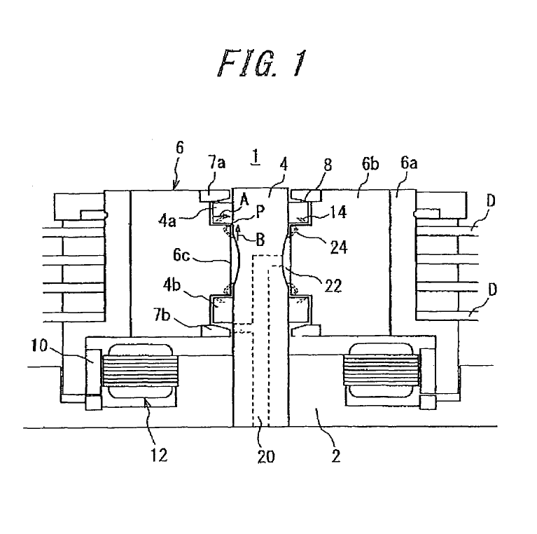

The fluid dynamic bearing and the method for lubricating the fluid dynamic bearing according to the invention will be described in detail below with reference to the accompanying drawing. FIG. 1 is a sectional view typically showing a schematic construction of a motor used for driving a recording disk and provided with a fluid dynamic bearing using a lubricating oil. In FIG. 1, a motor 1 comprises a bracket 2, a shaft 4 wherein one end thereof is extrapolated and fixed into a central opening of the bracket 2, and a rotor 6 held so as to be relatively rotatable around the shaft 4. A stator 12 is fixed to the bracket 2 and a rotor magnet 10 is positioned opposite the stator 12 and placed to the rotor 6, between which a rotary driving force are generated.

Further, an upper portion and a lower portion of the shaft 4 are provided with an upper thrust plate 4a and a lower thrust plate 4b which protrude outward in the radial direction and are in the form of a circular disc, and a gas intercalated portion 22 is formed on a lateral surface of the shaft between these thrust plates. On the other hand, the rotor 6 comprises a rotor hub 6a placing and holding a recording disc D on its outer peripheral portion, and a sleeve 6b arranged in an inner peripheral side of the rotor 6 and supported by the shaft 4 via a micro-gap holding a lubricating oil 8. Further, the sleeve 6b is provided with an upper counter plate 7a and a lower counter plate 7b which cover the outside of the upper and lower thrust plates.

In this context, a micro-gap is formed in a portion from an outer peripheral portion of the shaft 4 adjacent to an upper portion of the gas intercalated portion 22 disposed in the central portion of the shaft 4 to an undersurface, an outer peripheral surface and an outer peripheral portion of an upper surface of the upper thrust plate 4a and between an upper portion of an inner peripheral portion through-hole 6c of the opposing sleeve 6b and an undersurface of the upper counter plate 7a, and it holds the lubricating oil 8. Further, a spiral groove 14 generating dynamic pressure in the lubricating oil 8 with a rotation of the rotor 6 is formed on the undersurface of the upper thrust plate 4a, and it generates a bearing power for holding a rotor portion in the shaft line direction while the motor rotates and also pushes back the lubricating oil 8 in the direction of an arrow A. Moreover, an unbalance and herringbone-shaped groove 24 is formed in a lubricating oil holding portion on an inner surface of the sleeve 6b and in the upper portion of the inner peripheral portion through-hole 6c, and it generates a bearing power for holding a rotor portion in the radial direction while the motor rotates and also pushes up the lubricating oil 8 in the direction of an arrow B.

Due to the dynamic pressure of the lubricating oil 8 caused by these grooves, a pressure distribution formed in the lubricating oil 8 in the micro-gap has a maximum point at an undersurface and inner peripheral portion P of the upper thrust plate 4a. As a result, even if an air dissolved in the lubricating oil 8 becomes a bubble, the bubble is diffused and excluded outside of the inner peripheral portion P and reaches a lower gap portion of the gas intercalated portion 22 or a upper gap portion on the undersurface of the upper counter plate 7a. Further, these gap portions are communicated directly or through an atmosphere communicating hole 20 to the atmosphere and thereby the bubble is evacuated to the atmosphere, and as a result, the fluid dynamic bearing structure preventing a lubricating oil leak and having a high bearing power is accomplished.

Furthermore, the similar structure of the micro-gap, groove and lubricating oil holding portion is turned upside down and formed from the lower portion of the gas intercalated portion 22 arranged in the central portion of the shaft 4 to the lower thrust plate 4b and the lower counter plate 7b, and by such an lower dynamic pressure bearing portion, the rotor portion is further stably supported. In the fluid dynamic bearing having the present structure, a transpiration of the lubricating oil 8 in the outer peripheral direction due to a rotary centrifugal force is effectively prevented by the upper and lower counter plates 7a, 7b even at a high rotating speed of about 20000 revolutions per minute. Further, by using the above-mentioned lubricating oil composition in the fluid dynamic bearing having the present structure, the fluid dynamic bearing can be used over a wide temperature range, and a further high-speed and stable rotation can be accomplished.

### Examples

The following examples are given in illustration of the invention and are not intended as limitations thereof.

<Evaluation of the Base Oil>

For (A) a diester of 1,8-octanediol and 2-ethyl hexanoic acid,

(B) a diester of 1,9-nonanediol and 2-ethyl hexanoic acid,

(C) a diester of 1,10-decanediol and 2-ethyl hexanoic acid,

(D) a diester of 1,9-nonanediol and n-octanoic acid,

(E) a diester of 2,4-diethyl-1,5-pentanediol and n-octanoic acid,

(F) a diester of sebacic acid and 2-ethyl-1-hexanol, and

(G) a diester of azelaic acid and 2-ethyl-1-hexanol, a kinematic viscosity, a viscosity index, a total acid number, a total hydroxyl value, an evaporation amount, a pour point, low-temperature flowability and hydrolytic stability are measured according to the following methods. Results are shown in Table 1.

(1) Kinematic Viscosity and Viscosity Index

According to JIS K 2283, the kinematic viscosity is measured with using a Cannon-Fenske viscometer and further the viscosity index is calculated.

(2) Total Acid Number

The total acid number is measured according to JIS K 2501.

(3) Total Hydroxyl Value

The total hydroxyl value is measured according to JIS K 0070.

(4) Evaporation Amount

The evaporation amount is determined from a decreased amount by mass after 12 hours of storage at 120° C. by thermogravimetry (TG).

(5) Pour Point

The pour point is measured according to JIS K 2269.

(6) Low-Temperature Flowability

20 mL of test oil is charged into a 50 mL sample bottle and stood at −40° C. for 30 days, and then flowability (solidified state) of the test oil is observed. In a case that the test oil does not flow within 1 minute even if the sample bottle is turned upside down after it is stood for 30 days, such an oil is evaluated to be solidified.

(7) Hydrolytic Stability

After the test oil is stored at 93° C. for 20 days, the total acid number of the test oil is measured according to ASTM D2619 to evaluate hydrolytic stability.

TABLE 1

| | | | | Comparative | Comparative | Comparative | Comparative |
|---|---|---|---|---|---|---|---|
| | Example 1 | Example 2 | Example 3 | Example 1 | Example 2 | Example 3 | Example 4 |
| Test oil | (A) | (B) | (C) | (D) | (E) | (F) | (G) |
| Molecular weight | 399 | 413 | 427 | 413 | 413 | 427 | 413 |
| Kinematic viscosity at 40° C. | 9.85 | 10.9 | 12.1 | 11.1 | 9.73 | 11.6 | 10.4 |
| (mm²/s) | | | | | | | |
| Viscosity index | 115 | 122 | 130 | 170 | 128 | 155 | 145 |
| Total acid number | 0.01 | 0.01 | 0.01 | 0.01 | 0.01 | 0.01 | 0.01 |
| (mgKOH/g) | | | | | | | |
| Hydroxyl value (mgKOH/g) | 2 | 1 | 2 | 2 | 1 | 2 | 1 |
| Evaporation amount (mass %) | 2.0 | 1.7 | 1.1 | 1.5 | 1.6 | 0.8 | 1.8 |
| Pour point (° C.) | <−50 | <−50 | <−50 | 20 | <−50 | <−50 | <−50 |
| Low-temperature flowability | Flowable | Flowable | Flowable | Solidified | Solidified | Flowable | Flowable |
| Hydrolytic stability | 0.41 | 0.52 | 0.37 | — | — | 5.13 | 5.96 |
| [Total acid number after 20 | | | | | | | |
| days of storage at 93° C. | | | | | | | |
| (mgKOH/g)] | | | | | | | |

As seen from the Examples 1-3, a diester of a straight-chain dihydric alcohol having a hydroxyl group at both terminals and a branched-chain saturated monovalent fatty acid has a low pour point and also has good low-temperature flowability after long storage. While, as seen from the Comparative Example 1, a diester of a straight-chain dihydric alcohol having a hydroxyl group at both terminals and a straight-chain saturated monovalent fatty acid has a high pour point and has bad low-temperature flowability after long storage. Further, as seen from the Comparative Example 2, a diester of a branched-chain dihydric alcohol having a hydroxyl group at both terminals and a straight-chain saturated monovalent fatty acid has a low pour point but has bad low-temperature flowability after long storage. Furthermore, as seen from the Comparative Examples 3 and 4, a diester of a straight-chain divalent carboxylic acid having a carboxyl group at both terminals and a branched-chain saturated monohydric alcohol has bad hydrolytic stability.

<Evaluation of the Oxidation Stability of the Lubricating Oil for the Fluid Dynamic Bearing>

Then, a lubricating oil composition is prepared by using the above-described diester as a base oil and adding an additive shown in Table 2, and the oxidation stability of the resulting lubricating oil composition is evaluated according to Rotating Bomb Oxidation Test (RBOT) in JIS K 2514. Results are shown in Table 2.

TABLE 2

| | Base oil | | Additive (mass %) | | | | | | Oxidation |
|---|---|---|---|---|---|---|---|---|---|
| | | Content | | Amine-based | Amine-based | | | | stability |
| | | (mass | Phenol-based | antioxidant | antioxidant | Epoxy | Carbodiimide | Triazole | RBOT |
| | Type | %) | antioxidant *1 | (DPA) *2 | (PNA) *3 | compound *4 | Compound *5 | Compound *6 | (minute) |
| Example 4 | (A) | 99.5 | — | 0.5 | — | — | — | — | 3730 |
| Example 5 | (B) | 99.5 | — | 0.5 | — | — | — | — | 3499 |
| Example 6 | (B) | 99.5 | — | — | 0.5 | — | — | — | 3325 |
| Example 7 | (C) | 99.5 | — | 0.5 | — | — | — | — | 3450 |
| Comparative | (F) | 99.5 | — | 0.5 | — | — | — | — | 2362 |
| Example 5 | | | | | | | | | |
| Comparative | (F) | 99.5 | — | — | 0.5 | — | — | — | 2244 |
| Example 6 | | | | | | | | | |
| Comparative | (G) | 99.5 | — | 0.5 | — | — | — | — | 1811 |
| Example 7 | | | | | | | | | |
| Example 8 | (A) | 99.0 | 0.5 | 0.5 | — | — | — | — | 2950 |
| Example 9 | (B) | 99.0 | 0.5 | 0.5 | — | — | — | — | 2522 |
| Example 10 | (C) | 99.0 | 0.5 | 0.5 | — | — | — | — | 2720 |
| Comparative | (F) | 99.0 | 0.5 | 0.5 | — | — | — | — | 1465 |
| Example 8 | | | | | | | | | |
| Example 11 | (B) | 99.4 | — | 0.5 | — | 0.1 | — | — | 3530 |
| Example 12 | (B) | 99.4 | — | 0.5 | — | — | 0.1 | — | 3568 |
| Example 13 | (B) | 99.4 | — | 0.5 | — | — | — | 0.1 | 3822 |
| Example 14 | (B) | 99.3 | — | 0.5 | — | 0.1 | — | 0.1 | 3905 |
| Example 15 | (B) | 99.2 | — | 0.5 | — | 0.1 | 0.1 | 0.1 | 3800 |
| Example 16 | (B) | 98.7 | 0.5 | 0.5 | — | 0.1 | 0.1 | 0.1 | 2854 |

*1 Irganox L135T manufactured by Chiba Specialty Chemicals, octyl 3-(3,5-di-t-butyl-4-hydroxyphenyl)propionate
*2 Vanlube 81 manufactured by Vanderbilt Corporation, 4,4′-dioctyl diphenylamine
*3 Irganox L06 manufactured by Chiba Specialty Chemicals, octylphenyl-α-naphthylamine
*4 2-ethylhexyl glycidyl ether
*5 1,3-bis(2,6-diisopropylphenyl) carbodiimide
*6 Irgamet 39 manufactured by Chiba Specialty Chemicals, benzotriazole derivative

As seen from the Examples 4-16, the lubricating oil composition using as a base oil a diester of a straight-chain dihydric alcohol having a hydroxyl group at both terminals and a branched-chain saturated monovalent fatty acid has good oxidation stability. While, the lubricating oil composition in the Comparative Examples 5-8 using as a base oil a diester of a straight-chain divalent carboxylic acid having a carboxyl group at both terminals and a branched-chain saturated monohydric alcohol has worse oxidation stability as compared with the lubricating oil composition in the Examples.

Further, as seen from a comparison of the Examples 4, 5 and 7 with the Examples 8, 9 and 10, when the amine-based antioxidant is used, high improvement in the oxidation stability can be accomplished by not adding the phenol-based antioxidant. Furthermore, as seen from the Examples 11-15, the oxidation stability of the lubricating oil can be further improved by adding the amine-based antioxidant and further adding at least one of an epoxy compound, a carbodiimide compound and a triazole compound.

<Evaluation of the Hydrolytic Stability of the Lubricating Oil for the Fluid Dynamic Bearing>

Then, a lubricating oil composition is prepared by using the above-described diester as a base oil and adding an additive shown in Table 3, and the hydrolytic stability of the resulting lubricating oil composition is evaluated. Results are shown in Table 3. In this context, *2, *4 and *5 in Table 3 are the same meaning as mentioned above.

TABLE 3

| | | | | | | Hydrolytic |
|---|---|---|---|---|---|---|
| | | | | | | stability |
| | | | Additive (mass %) | | | Total acid number |
| | Base oil | | Amine-based | Epoxy | Carbodiimide | after 20 days of |
| | | Content | antioxidant | compound | Compound | storage at 93° C. |
| | Type | (mass %) | (DPA) *2 | *4 | *5 | (mgKOH/g) |
| Example 2 | (B) | 100.0 | — | — | — | 0.52 |
| Example | (B) | 99.9 | — | 0.1 | — | 0.12 |
| 17 | | | | | | |
| Example | (B) | 98.9 | 0.5 | 0.1 | 0.5 | 0.04 |
| 18 | | | | | | |

As seen from Table 3, the hydrolytic stability of the diester base oil is improved by adding an epoxy compound or a carbodiimide compound.

1. A lubricating oil composition comprising a base oil of a diester, wherein the diester is obtained from a straight-chain dihydric alcohol having a carbon number of 6 to 12 and having a hydroxyl group at both terminal carbon atoms respectively and a branched-chain saturated monovalent fatty acid having a carbon number of 6 to 12.

2. A lubricating oil composition according to claim 1, wherein the dihydric alcohol is 1,8-octanediol, 1,9-nonanediol or 1,10-decanediol.

3. A lubricating oil composition according to claim 1, wherein the monovalent fatty acid is 2-ethyl hexanoic acid or 3,5,5-trimethyl hexanoic acid.

4. A lubricating oil composition according to claim 1, comprising a viscosity index of not lower than 100 and a pour point of not higher than −50° C.

5. A lubricating oil composition according to claim 1, comprising 0.01 to 5% by mass of an amine-based antioxidant.

6. A lubricating oil composition according to claim 5, wherein a content of a phenol-based antioxidant is not more than 0.1% by mass.

7. A lubricating oil composition according to claim 1, comprising at least one selected from the group consisting of an epoxy compound, a carbodiimide compound and a triazole compound in an amount of 0.01 to 2% by mass.

8. A lubricating oil for a fluid dynamic bearing, which is composed of a lubricating oil composition as claimed in claim 1.

9. A fluid dynamic bearing comprising a shaft and a sleeve, wherein a lubricating oil composition as claimed in claim 1 is held in a gap between the shaft and the sleeve.

10. A method for lubricating a fluid dynamic bearing comprising a shaft and a sleeve, which lubricates a gap between the shaft and the sleeve by using a lubricating oil composition as claimed in claim 1.
